(19) United States
(12) Patent Application Publication
(10) Pub. No.: US 2021/0040213 A1
(43) Pub. Date: Feb. 11, 2021

(54) IMMUNOMONOTHERAPY FOR UROTHELIAL CARCINOMA

(71) Applicant: BEIGENE, LTD., Camana Bay, Grand Cayman (KY)

(72) Inventors: James SONG, Cambridge, MA (US); Yun ZHANG, Cambridge, MA (US); Zhirong SHEN, Beijing (CN); Qinzhou QI, Beijing (CN)

(21) Appl. No.: 16/967,840

(22) PCT Filed: Feb. 8, 2019
(86) PCT No.: PCT/US19/17313

§ 371 Date: Aug. 6, 2020

Related U.S. Application Data
(60) Provisional application No. 62/628,648, filed on Feb. 9, 2018.

(30) Foreign Application Priority Data
Feb. 10, 2018 (CN) PCT/CN2018/076100

(51) Int. Cl. C07K 16/28; A61K 39/395; A61K 33/243; A61K 31/7068; A61K 31/337; A61P 35/00

(52) U.S. Cl. CPC C07K 16/2818; A61K 39/39541; A61K 33/243; A61K 31/7068; A61K 31/337; A61K 2039/545; C07K 2317/71; C07K 2317/51; C07K 2317/56; C07K 2317/52; A61P 35/00

(57) ABSTRACT

Disclosed herein is a method for immunotherapy of a patient with urothelial carcinoma (UC) comprising administering to the patient an anti-PD-1 antibody reduces FcγR binding thus reducing antibody-dependent phagocytosis.

### CROSS REFERENCE TO RELATED APPLICATIONS

This application claims the benefit of U.S. Provisional Application No. 62/628,648 filed on Feb. 9, 2018 and International Patent Application No. PCT/CN2018/076100 filed on Feb. 10, 2018, the disclosures of each of which are hereby incorporated by reference in their entireties for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: BEIG_034_01WO_SeqList.TXT, date recorded Feb. 8, 2019, file size 126 kilobytes).

### FIELD OF THE INVENTION

Disclosed herein is a method of treatment of a patient with urothelial carcinoma (UC) comprising administering to the patient an anti-PD-1 antibody which was specifically engineered to reduce FcγR binding on macrophages to abrogate antibody-dependent phagocytosis.

### BACKGROUND OF THE INVENTION

Urothelial carcinoma is also known as urothelial (transitional cell) carcinoma (UC) or urothelial carcinoma (transitional cell tumors, or transitional cell tumors of the urothelium), as well as bladder cancer. More than 90% of urothelial tumors originate in the urinary bladder, 8% originate in the renal pelvis, and the remaining 2% originate in the ureter and urethra.

Platinum-based combination chemotherapy has been used for decades as the first-line therapy for patients suffering from advanced urothelial carcinoma. For example, the standard of care in first-line treatment of bladder cancer includes the combination chemotherapy of methotrexate, vinblastine, doxorubicin, and cisplatin (MVAC), which has been shown to have considerable toxicity. Another combination chemotherapy is cisplatin-gemcitabine combination which has replaced MVAC as the standard for first-line treatment of metastatic bladder cancer, and 90% of the patients that have reported relapse have very poor prognosis. Advances in treatment of metastatic UC have stagnated over the past 3 decades.

WO2016064649A1 discloses a combination therapy of an anti-VEGF receptor 2 antibody (ramucirumab) in simultaneous, separate or sequential combination with docetaxel to treat patients with UC. This indicates that antibody therapies are effective agents against UC.

Monoclonal antibodies (mAbs) against immune checkpoint inhibitory receptors, like programmed cell death-1 (PD-1), have demonstrated promising antitumor activity across multiple malignancies (Topalian S L, et al. N Engl J Med. 2012; 366: 2443-2454.), including UC (Plimack E R, et al. J Clin Oncol. 2015; 33(suppl; abstr 4502); Bellmunt J, et al. Abstract 470. Presented at 31st Society for Immunotherapy of Cancer Annual Meeting, Nov. 9-13,2016, National Harbor, Md., USA; Balar A, et al. Ann Oncol. 2016; Volume 27, Issue suppl_6, 1 Oct. 2016, LBA32_PR.; Sharma P, et al. J Clin Oncol. 2016; 34(supplemental abstract 4501); Galsky M D, et al. J Clin Oncol. 2016; LB: A31; and Sharma P, et al. Lancet Oncol. 2016; 17: 1590-1598).

PD-1 is relatively overexpressed on CD8⁺ effector, tumor-infiltrating T lymphocytes (TILs); and anti-PD-1 antibodies induce an increase in CD8⁺ T cell percentages within the tumor microenvironment (Ahmadzadeh M, et al., Blood 2009;114: 1537-1544).

WO 2015/035606 Al discloses a humanized IgG4 mAb with high affinity and binding specificity against PD-1, in particular a monoclonal antibody which specifically binds to PD-1, including residues K45 and I93; or, I93, L95 and P97, and inhibits PD-1-mediated cellular signaling in immune cells, by binding to a set of amino acid residues required for its ligand (PD-L1) binding. WO 2015/035606 discloses a monoclonal antibody which binds human PD-1, and a modified IgG4 Fc region, wherein the modified IgG4 Fc region reduces binding of the antibody to one or more Fcγ receptors.

Dahan R, et al., reported in vivo evidence that anti-PD-1 antibodies demonstrate reduced tumor cytotoxicity when the Fc domain of the antibody engages with Fc⁻gamma receptors (FcγRs) (Dahan R, et al. Cancer Cell. 2015; 28: 285-295). FcγR engagement has been reported to result in preferential depletion of these CD8⁺ TILs. This decrease can correlate with the reduction of anti-PD-1 tumor activity.

The antibodies of WO 2015/035606 A1 were specifically engineered to reduce FcγR binding on macrophages and myeloid-derived suppressor cells; reduction of FcγR binding can improve clinical activity by preserving activated T cells and this proposed mechanism of action is shown graphically in FIG. 1.

As the advances in the treatment of UC have slowed, the inventors have found that the antibodies of the current disclosure can be useful as a new immunotherapy for urothelial carcinoma including advanced or metastatic urothelial carcinoma.

### SUMMARY OF THE INVENTION

Disclosed herein is a method of treatment of a patient with urothelial carcinoma (UC) comprising administering to the patient a therapeutically effective amount of an anti-PD-1 antibody or an antigen binding fragment thereof, which was specifically engineered to reduce FcγR binding on macrophages to reduce antibody-dependent phagocytosis.

In one embodiment, the urothelial carcinoma involves the ureter, urethra, renal pelvis and/or bladder. In another embodiment, the urothelial carcinoma is transitional cell carcinoma. In yet another embodiment, the urothelial carcinoma is advanced or metastatic.

In one embodiment, the patient is suffering from PD-L1⁺ urothelial carcinoma or PD-L1⁻ urothelial carcinoma. In another embodiment, the patient is suffering from PD-L1⁺ urothelial carcinoma.

In one embodiment, the anti-PD-1 antibody is the one disclosed in WO 2015/035606 A1. The antibodies disclosed in WO 2015/035606 A1 specifically bind to Programmed Death-1 (PD-1) receptor and inhibit PD-1-mediated cellular signaling in immune cells, antibodies binding to a set of amino acid residues required for its ligand binding. In another embodiment, the anti-PD-1 antibody is a humanized monoclonal antibody comprising a heavy chain variable region (V_{H}) and a light chain variable region (V_{L}) (comprising SEQ ID NO: 24 and SEQ ID NO: 26, respectively) and a IgG4 heavy chain effector or constant domain (SEQ ID NO: 88), hereinafter known Mab-1, which specifically binds to PD-1, including PD-1 receptor residues K45 and I93; or, I93, L95 and P97, and inhibits PD-1-mediated cellular signaling in immune cells.

In other embodiment, the anti-PD-1 antibody is a monoclonal antibody which binds human PD-1, comprising a modified IgG4 Fc region, wherein the modified IgG4 Fc region reduces binding of the antibody to one or more Fcγ receptors. In a preferred embodiment, the anti-PD-1 antibody is a monoclonal antibody which binds human PD-1, comprising an IgG4 Fc region comprising a S228P mutation at position 228 and amino acid mutations at positions 233, 234, and 235, wherein the mutations at positions 233, 234, and 235 cause the antibody to exhibit reduced binding to at least one Fcγ receptor relative to Fc binding of a reference IgG4 antibody having a mutation only at position 228 and no other Fc region mutation as disclosed in WO 2015/035606 A1. In a further embodiment, the IgG4 Fc region comprises amino acid mutations at positions 228, 233, 234, 235, and 265 as disclosed in WO 2015/035606 A1. In a yet another embodiment, the IgG4 Fc region comprises amino acid mutations at positions 228, 233, 234, 235, 265, 309, and 409 as disclosed in WO 2015/035606 A1. In another embodiment, the IgG4 Fc region comprises amino acid mutations of S228P, E233P, F234V, and L235A as disclosed in WO 2015/035606 A1. In one embodiment, the above-mentioned antibody may further comprise an IgG4 heavy chain constant domain comprising any of SEQ ID NOs: 83-88.

The antibody disclosed herein, i.e., Mab-1, has previously shown to be tolerable in patients with advanced solid tumors such as hepatocellular carcinoma and its toxicity profile demonstrates that adverse events (AEs) are generally of low-to-moderate severity, manageable, and reversible; Desai J, et al., J Immunother Cancer 2016; 4(Suppl 1): P154; and WO2019/001417 published Jan. 3, 2019).

The method of treatment as disclosed herein has been shown in human patients to reduce, delay the progression of and alleviate urothelial carcinoma. The inventors of the present disclosure have found that an anti-PD-1 antibody, for example, Mab-1, can be efficacious as immunotherapy for treating urothelial carcinoma, especially PD-L1⁺ urothelial carcinoma as objective responses were observed at a higher rate in PD-L1⁺ UC compared with PD-L1⁻ UC. The inventors also found that the treatment with Mab-1 is generally well tolerated in pretreated patients with UC, and the adverse events reported in patients with UC were consistent with the overall safety profile observed in the study and were generally of low or moderate severity, manageable, and reversible.

### BRIEF DESCRIPTION OF THE DRAWINGS

FIG. 1 shows a potential mechanism of T-cell clearance of an anti-PD-1 antibody used in the present application, i.e., reduction of FcγR binding prevents macrophage-mediated T-cell clearance.

FIG. 2 shows a schematic of the study design.

FIG. 3 shows the duration of treatment and response in patients with UC treated with the antibody disclosed herein.

FIG. 4 shows maximum tumor reduction in patients with UC treated with the antibody disclosed herein.

FIG. 5 shows the change in target lesions over time in patients with UC treated with the antibody disclosed herein.

FIG. 6A-6H are radiographic images of a 74-year-old male with PD-L1 positive UC who had previously failed three prior anti-cancer treatments that did not include immunotherapy treatment of any kind. The images show regression of the UC tumor from day 0 (baseline) to 46 cycles of treatment.

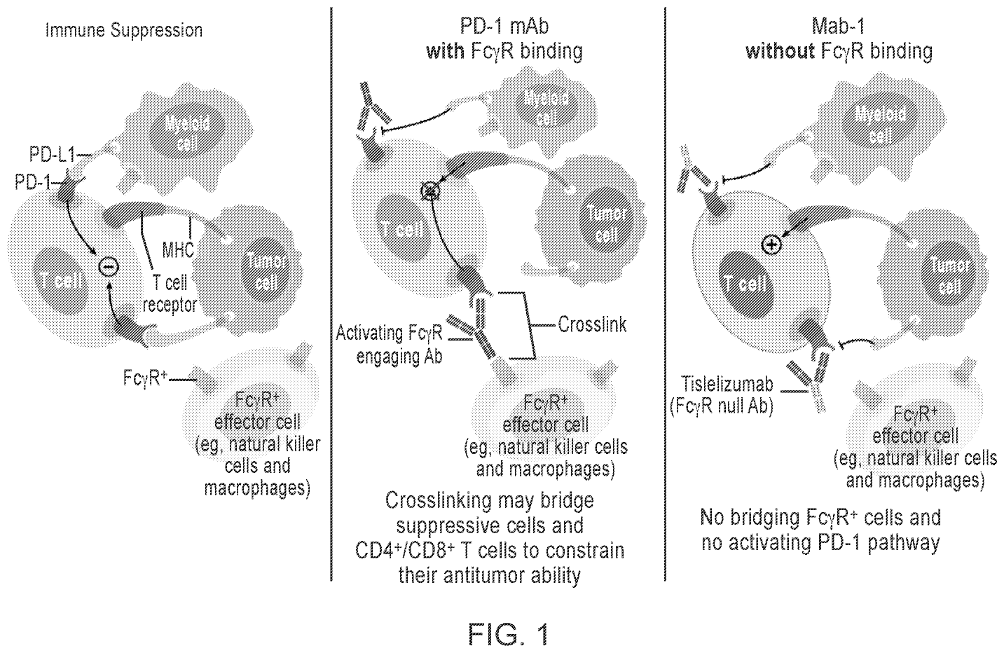

### DETAILED DESCRIPTION OF THE INVENTION

### Abbreviations

Throughout the detailed description and examples disclosed herein, the following abbreviations will be used:
Ab antibody
AE Adverse event
CD cluster of differentiation
CDR Complementarity determining region
CR complete response
DPBS Dulbecco's Phosphate Buffered Saline
FcγR Fc-gamma receptor
i.p. Intraperitoneal or Intraperitoneally
i.v. intravenous or intravenously
IFN-γ Interferon-γ
IgG immunoglobulin G
mAb monoclonal antibody
max maximum
MHC major histocompatibility complex
min minimum
NK Natural killer
p.o. “by mouth” or “per os”
PD progressive disease
PD-1 Programmed Death 1 protein, Pdcd-1, or CD279
PD-L1 programmed cell death ligand-1
PDX Patient-derived xenograft
PR partial response
Q2W once every 2 weeks
Q3W once every 3 weeks
Q4W Once every four weeks
QW Once weekly
RP2D recommended Phase 2 dose
SD stable disease
TILs Tumor-infiltrating lymphocytes
UC urothelial carcinoma
V_{H} Heavy chain variable region
V_{L} Light chain variable region

### Definitions

Unless specifically defined elsewhere in this document, all other technical and scientific terms used herein have the meaning commonly understood by one of ordinary skill in the art to which this invention belongs.

As used herein, including the appended claims, the singular forms of words such as “_{Li}” “an,” and “the” include their corresponding plural references unless the context clearly dictates otherwise.

The term “antibody” herein is used in the broadest sense and specifically covers antibodies (including full length monoclonal antibodies) and antibody fragments so long as they recognize PD-1. An antibody molecule is usually monospecific, but may also be described as idiospecific, heterospecific, or polyspecific. Antibody molecules bind by means of specific binding sites to specific antigenic determinants or epitopes on antigens.

The term “monoclonal antibody” or “mAb” herein means a population of substantially homogeneous antibodies, i.e., the antibody molecules comprised in the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. Monoclonal antibodies (mAbs) may be obtained by methods known to those skilled in the art. See, for example, Kohler and Milstein, Nature (London) 256:495 (1975); U.S. Pat. No. 4,376,110; Ausubel al, Short Protocols in Molecular Biology, 3rd ed., Wiley & Sons, 1995; Harlow & Lane, Using Antibodies, A Laboratory Manual (1998); and Colligan et al., eds., Current Protocols in Immunology, Greene Publishing Assoc. and Wiley Interscience, N.Y., (1992, 1993). The mAbs disclosed herein may be of any immunoglobulin class including IgG, IgM, IgD, IgE, IgA, and any subclass thereof. A hybridoma producing a mAb may be cultivated in vitro or in vivo. High titers of mAbs can be obtained in in vivo production where cells from the individual hybridomas are injected intraperitoneally into mice, such as pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (V_{L}\V_{H}) pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called “complementarity determining regions (CDRs),” which are located within relatively conserved framework regions (FR). The CDRs are usually aligned by the framework regions, enabling binding to a specific epitope. In general, from N-terminal to C-terminal, both light and heavy chains variable domains comprise FR-1, CDR-1, FR-2, CDR-2, FR-3, CDR-3, and FR-4. The assignment of amino acids to each domain is, generally, in accordance with the definitions of Sequences of Proteins of Immunological Interest, Kabat, et al. National Institutes of Health, Bethesda, Md., 5th ed., NIH Publ. No. 91-3242 (1991); Kabat (1978) Adv. Prot. Chem. 32: 1-75; Kabat, et al., (1977) J. Biol. Chem. 252:6609-6616; Chothia, et al, (1987) J Mol. Biol. 196:901-917 or Chothia, et al, (1989) Nature 342:878-883.

The term “hypervariable region” means the amino acid residues of an antibody that are responsible for antigen-binding. The hypervariable region comprises amino acid residues from a “complementarity determining region” or “CDR” (i.e., CDR-L1, CDR-L2 and CDR-L3 in the light chain variable domain and CDR-H1, CDR-H2 and CDR-H3 in the heavy chain variable domain). See, Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues. Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

Unless otherwise indicated, “antibody fragment” or “antigen binding fragment” means antigen binding fragments of antibodies, i.e. antibody fragments that retain the ability to bind specifically to the antigen bound by the full-length antibody, e.g. fragments that retain one or more CDR regions. Examples of antibody binding fragments include, but not limited to, Fab, Fab′, F(ab′)2, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. An antibody herein is said to bind specifically to a polypeptide comprising a given amino acid sequence, e.g. the amino acid sequence of a mature human PD-1 molecule, if it binds to polypeptides comprising that sequence but does not bind to proteins lacking that sequence.

The term “human antibody” herein means an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin sequences, respectively.

The term “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu” or “h” is added to antibody clone designations when necessary to distinguish humanized antibodies from parental rodent antibodies. The humanized forms of rodent antibodies can comprise the same CDR sequences of the parental rodent antibodies, although certain amino acid substitutions can be included to increase affinity, increase stability of the humanized antibody, or for other reasons.

The term “treatment” or “treatin2” is an approach for obtaining beneficial or desired clinical results, including, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Therefore, a reduction of pathological consequence of urothelial carcinoma is also included by the term “treatment.” The methods disclosed herein encompass any one or more of these aspects of treatment.

### Anti-PD-1 Antibody

As disclosed herein, the anti-PD-1 antibody is an antibody or an antigen binding fragment thereof, which specifically binds to human PD-1.

As disclosed herein, the anti-PD-1 antibody is an antibody which comprises a heavy chain variable region (V_{H}) and a light chain variable region (V_{L}) that contain complementarity determining region(s) (CDRs) which are defined using the Kabat numbering system and listed as follows:

| | |
|---|---|
| a) mu317 | CDR-H1, CDR-H2 and CDR-H3 (SEQ ID NOs: 11, 12, 13, respectively); and |
| | CDR-L1, CDR-L2 and CDR-L3 (SEQ ID NOs: 14, 15, 16, respectively); |
| b) mu326 | CDR-H1, CDR-H2 and CDR-H3 (SEQ ID NOs: 17, 18, 19, respectively); and |
| | CDR-L1, CDR-L2 and CDR-L3 (SEQ ID NOs: 20, 21, 22, respectively); |
| c) 317-4B6 | CDR-H1, CDR-H2 and CDR-H3 (SEQ ID NOs: 31, 32, 33, respectively); and |
| | CDR-L1, CDR-L2 and CDR-L3 (SEQ ID NOs: 34, 35, 36, respectively); |
| d) 326-4A3 | CDR-H1, CDR-H2 and CDR-H3 (SEQ ID NOs: 37, 38, 39, respectively); and |
| | CDR-L1, CDR-L2 and CDR-L3 (SEQ ID NOs: 40, 41, 42, respectively); |
| e) 317-1H | CDR-H1, CDR-H2 and CDR-H3 (SEQ ID NOs: 11, 59, 13, respectively); and |
| | CDR-L1, CDR-L2 and CDR-L3 (SEQ ID NOs: 14, 15, 16, respectively); |
| f) 317-4B2 | CDR-HL CDR-H2 and CDR-H3 (SEQ ID NOs: 11, 60, 13, respectively); and |
| | CDR-L1, CDR-L2 and CDR-L3 (SEQ ID NOs: 61,15, 16, respectively); |
| g) 317-4B5 | CDR-H1, CDR-H2 and CDR-H3 (SEQ ID NOs: 11,60, 13, respectively); and |
| | CDR-L1, CDR-L2 and CDR-L3 (SEQ ID NOs: 61,15, 16, respectively); |
| h) 317-4B6 | CDR-H1, CDR-H2 and CDR-H3 (SEQ ID NOs: 11, 32, 13, respectively); and |
| | CDR-L1, CDR-L2 and CDR-L3 (SEQ ID NOs: 61, 15, 16, respectively); |
| i) 326-1 | CDR-H1, CDR-H2 and CDR-H3 (SEQ ID NOs: 17, 62, 19, respectively); and |
| | CDR-L1, CDR-L2 and CDR-L3 (SEQ ID NOs: 20, 21, 22, respectively); |
| j) 326-3B1 | CDR-H1, CDR-H2 and CDR-H3 (SEQ ID NOs: 17, 62, 19, respectively); and |
| | CDR-L1, CDR-L2 and CDR-L3 (SEQ ID NOs: 20, 21, 22, respectively); |
| or k) 326-3G1 | CDR-H1, CDR-H2 and CDR-H3 (SEQ ID NOs: 17, 62, 19, respectively); and |
| | CDR-L1, CDR-12 and CDR-L3 (SEQ ID NOs: 20, 21, 22, respectively). |

As disclosed herein, the anti-PD-1 antibody is an antibody which comprises a heavy chain variable region (V_{H}) and a light chain variable region (V_{L}) that contain any combinations of CDRs listed as follows:

| | |
|---|---|
| (a) | CDR-H1 (SEQ ID NO 31), CDR-H2 (SEQ ID NO 12, 32, 59 or 60) |
| | and CDR-H3 (SEQ ID NO 33),CDR-L1 ( SEQ ID NO 14, 34 or 61), |
| | CDR-L2 (SEQ ID NO 35) and CDR-L3 (SEQ ID NO 36); or |
| (b) | CDR-H1 (SEQ ID NO 37), CDR-H2 (SEQ ID NO 18, 38 or 62) |
| | and CDR-H3 (SEQ ID NO 39),CDR-L1 (SEQ ID NO 40), |
| | CDR-L2 (SEQ ID NO 41) and CDR-L3 (SEQ ID NO 42). |

As disclosed herein, the anti-PD-1 antibody is an antibody which comprises a heavy chain variable region (V_{H}) and a light chain variable region (V_{L}) comprising:

a) mu317 (SEQ ID NOs: 4 and 6, respectively);
b) mu326 (SEQ ID NOs: 8 and 10, respectively);
c) 317-4B6 (SEQ ID NOs: 24 and 26, respectively);
d) 326-4A3 (SEQ ID NOs: 28 and 30, respectively);
e) 317-4B2 (SEQ ID NOs: 43 and 44, respectively);
f) 317-4B5 (SEQ ID NOs: 45 and 46, respectively);
g) 317-1 (SEQ ID NOs: 48 and 50, respectively);
h) 326-3B1 (SEQ ID NOs: 51 and 52, respectively);
i) 326-3GI (SEQ ID NOs: 53 and 54, respectively);
j) 326-1 (SEQ ID NOs: 56 and 58, respectively);
k) 317-3A1 (SEQ ID NOs: 64 and 26, respectively);
l) 317-3C1 (SEQ ID NOs: 65 and 26, respectively);
m) 317-3E1 (SEQ ID NOs: 66 and 26, respectively);
n) 317-3F1 (SEQ ID NOs: 67 and 26, respectively);
o) 317-3G1 (SEQ ID NOs: 68 and 26, respectively);
p) 317-3H1 (SEQ ID NOs: 69 and 26, respectively);
q) 317-311 (SEQ ID NOs: 70 and 26, respectively);
r) 317-4B 1 (SEQ ID NOs: 71 and 26, respectively);
s) 317-4B3 (SEQ ID NOs: 72 and 26, respectively);
t) 317-4B4 (SEQ ID NOs: 73 and 26, respectively);
u) 317-4A2 (SEQ ID NOs: 74 and 26, respectively);
v) 326-3 A 1 (SEQ ID NOs: 75 and 30, respectively);
w) 326-3C1 (SEQ ID NOs: 76 and 30, respectively);
x) 326-3D1 (SEQ ID NOs: 77 and 30, respectively);
y) 326-3E1 (SEQ ID NOs: 78 and 30, respectively);
z) 326-3F1 (SEQ ID NOs: 79 and 30, respectively);
aa) 326-3B N55D (SEQ ID NOs: 80 and 30, respectively);
ab) 326-4A1 (SEQ ID NOs: 28 and 81, respectively); or
ac) 326-4A2 (SEQ ID NOs: 28 and 82, respectively).

In some embodiments, the antibody comprises an IgG4 Fc region having a serine to proline mutation at position 228 (EU numbering system). In some embodiments, this mutation is referred to as the S228P mutation. In some embodiments, the antibody comprises an IgG4 Fc region having a mutation at one or more of positions 233, 234, 235, 265, 309, and 409 (EU numbering system). For example, in some embodiments, the antibody comprises an IgG4 region having a mutation at 228 and at least one other position, wherein the at least one other mutation results in reduced binding to one or more FcγR. In further embodiments, the antibody comprises an IgG4 region having a mutation at position 228 and at least two, at least 3, at least 4, at least 5, or at least 6 additional positions, wherein one or more of the additional mutations results in reduced binding to one or more FcγR. In some embodiments, the antibody comprises an IgG4 region having mutations at positions 234 and 235. In some embodiments, the antibody comprises an IgG4 region having mutations at positions 233, 235, and 235. In some embodiments, the antibody comprises an IgG4 region having mutations at positions 234, 235, and 265. In some embodiments, the antibody comprises an IgG4 region having mutations at positions 233, 234, 235, and 265. In some embodiments, the antibody comprises an IgG4 region having mutations at positions 234, 235, 265, and 409. In some embodiments, the antibody comprises an IgG4 region having mutations at positions 233, 234, 235, 265, and 409. In some embodiments, the antibody comprises an IgG4 region having mutations at positions 234, 235, 265, 309, and 409. In some embodiments, the antibody comprises an IgG4 region having mutations at positions 233, 234, 235, 265, 309, and 409. The mutation at position 234 may be a phenylalanine to valine substitution or a phenylalanine to alanine substitution. The mutation at position 235 may be a leucine to alanine substitution. The mutation at position 233 may be a glutamic acid to proline substitution. The mutation at position 265 may be a aspartic acid to valine substitution or an aspartic acid to threonine substitution. The mutation at position 309 may be a leucine to valine substitution. The mutation at position 409 may be an arginine to a lysine, threonine, or methionine substitution.

As disclosed herein, the anti-PD-1 antibody comprises a IgG4 heavy chain constant domain comprising any of SEQ ID NOs: 83-88 or 91-106.

As disclosed above, the anti-PD-1 antibody is an antibody which contains a F(ab) or F(ab′)2 comprising a domain said above, including a heavy chain variable region (V_{H}), a light chain variable region (V_{L}) and an IgG4 heavy chain constant domain.

As disclosed herein, the anti-PD-1 antibody comprises a heavy chain variable region (V_{H}) and a light chain variable region (V_{L}), and a IgG4 heavy chain constant domain comprising SEQ ID NOs: 87 or 88, wherein the heavy chain variable region (V_{H}) and the light chain variable region (V_{L}) comprise:

a) mu317 (SEQ ID NOs: 4 and 6, respectively);
b) mu326 (SEQ ID NOs: 8 and 10, respectively);
c) 317-4B6 (SEQ ID NOs: 24 and 26, respectively);
d) 326-4A3 (SEQ ID NOs: 28 and 30, respectively);
e) 317-4B2 (SEQ ID NOs: 43 and 44, respectively);
f) 317-4B5 (SEQ ID NOs: 45 and 46, respectively);
g) 317-1 (SEQ ID NOs: 48 and 50, respectively);
h) 326-3B1 (SEQ ID NOs: 51 and 52, respectively);
i) 326-3GI (SEQ ID NOs: 53 and 54, respectively);
j) 326-1 (SEQ ID NOs: 56 and 58, respectively);
k) 317-3A1 (SEQ ID NOs: 64 and 26, respectively);
l) 317-3C1 (SEQ ID NOs: 65 and 26, respectively);
m) 317-3E1 (SEQ ID NOs: 66 and 26, respectively);
n) 317-3F1 (SEQ ID NOs: 67 and 26, respectively);
o) 317-3G1 (SEQ ID NOs: 68 and 26, respectively);
p) 317-3H1 (SEQ ID NOs: 69 and 26, respectively);
q) 317-311 (SEQ ID NOs: 70 and 26, respectively);
r) 317-4B 1 (SEQ ID NOs: 71 and 26, respectively);
s) 317-4B3 (SEQ ID NOs: 72 and 26, respectively);
t) 317-4B4 (SEQ ID NOs: 73 and 26, respectively);
u) 317-4A2 (SEQ ID NOs: 74 and 26, respectively);
v) 326-3 A 1 (SEQ ID NOs: 75 and 30, respectively);
w) 326-3C1 (SEQ ID NOs: 76 and 30, respectively);
x) 326-3D1 (SEQ ID NOs: 77 and 30, respectively);
y) 326-3E1 (SEQ ID NOs: 78 and 30, respectively);
z) 326-3F1 (SEQ ID NOs: 79 and 30, respectively);
aa) 326-3B N55D (SEQ ID NOs: 80 and 30, respectively);
ab) 326-4A1 (SEQ ID NOs: 28 and 81, respectively); or
ac) 326-4A2 (SEQ ID NOs: 28 and 82, respectively).

As disclosed herein, the anti-PD-1 antibody comprises a heavy chain variable region (V_{H}) and a light chain variable region (V_{L}), and an IgG4 heavy chain effector or constant domain comprising SEQ ID NO: 88, wherein the heavy chain variable region (V_{H}) and the light chain variable region (V_{L}) comprises SEQ ID NO: 24 and SEQ ID NO: 26, respectively.

As disclosed herein, the anti-PD-1 antibody contains a uniquely engineered humanized IgG4 Fc domain which reduces FcγR to reduce antibody-dependent phagocytosis, a potential mechanism of T-cell clearance, which results in increased effectiveness.

The Anti-PD1 Antibodies and antigen binding fragments thereof may be prepared as disclosed in WO 2015/035606 A1, which is incorporated herein by reference.

### Methods of Treatment

In the methods of treatment disclosed herein, the Mab-1 antibody at a certain dose was administered to the patients with UC. In one embodiment, the patients with UC are not treated by prior immunotherapy molecules. In another embodiment, the patient with UC was treated with an anti-cancer systemic therapy such as MVAC. In another embodiment, the patient had prior platinum adjuvant and neoadjuvant therapies.

In the methods of treatment for UC, the anti-PD-1 antibody is administered at a dose of 0.5-10 mg/kg QW, or Q2W, or Q3W, or Q4W. In some embodiments herein, Mab-1 is administrated at a dose of 0.5-10 mg/kg QW or Q2W or Q3W. In another treatment regimen, Mab-1 is administrated at a dose of 2-10 mg/kg Q2W or Q3W or is administered at a fixed dose of 200 mg Mab-1. Mab-1 can also be administrated intravenously at a dose of 2.0 mg/kg Q2W, 5 mg/kg Q2W, 2 mg/kg Q3W, or 5 mg/kg Q3W or at a fixed dose of 200 mg of Mab-1.

In another embodiment, treatment of UC with Mab-1 is in combination with another pharmaceutical agent or agents. In one embodiment, treatment of UC with Mab-1 is in combination with the standard of care for UC, that of methotrexate, vinblastine, doxorubicin, and cisplatin (MVAC). In yet another embodiment, treatment of UC is with Mab-1 and cisplatin or cisplatin and gemcitabine. A variation of that treatment is administration of paclitaxel prior to the cisplatin and gemcitabine, with Mab-1 being administered either before or during the paclitaxel treatment and before or during the cisplatin and gemcitabine treatment.

### EXAMPLES

### Study Design-Patient Differentiation and Enrollment

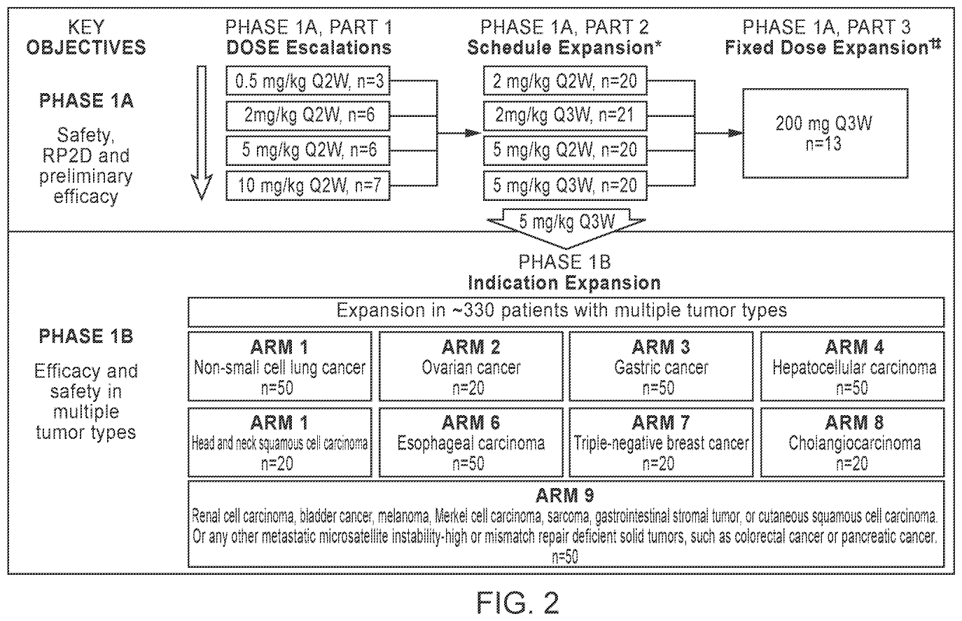

The purpose of the study design was to enroll UC patients for dosage determination and preliminary patient differentiation, during phase 1A and phase 1B. The study design is detailed in FIG. 2. In FIG. 2, * shows the schedule of Dose Expansion; while † indicates fixed doses that do not exceed the exposure of maximum tolerated dose, and indicates ‡ conducted in parallel with Phase 1B.

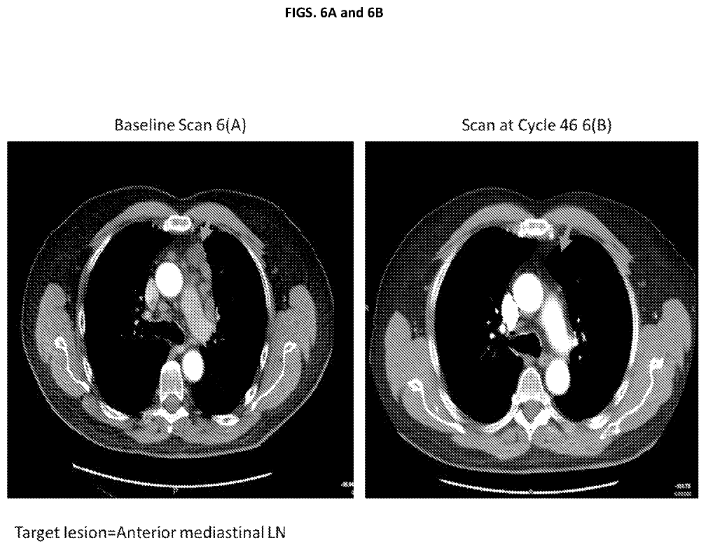

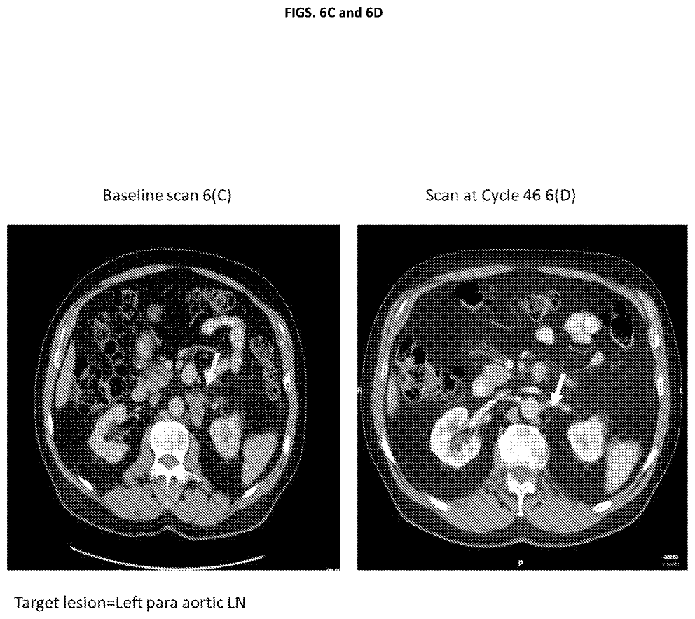

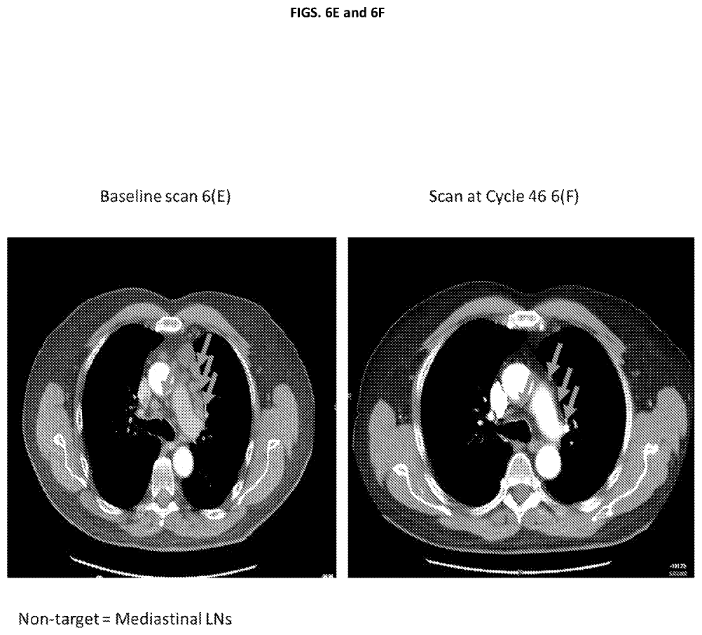

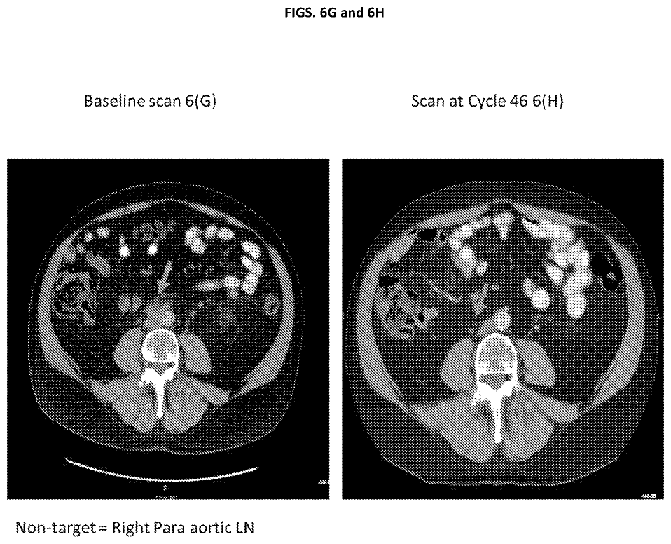

Phase 1A was used to determine safety, RP2D and preliminary efficacy of Mab-1. In Phase 1A, 10 mg/kg once every 2 weeks (Q2W) was the maximum administered dosage of Mab-1 and the maximum tolerated dose (MTD) was not reached. In Phase 1A, Part 1, a study of dose escalations starting from 0.5 mg/kg Q2W to 10 mg/kg Q2W was conducted. In Phase 1A, Part 2, a study of schedule expansion was conducted with 2 or 5 mg/kg of Mab-1 Q2W or Q3W. In Phase 1A, Part 3, a study of fixed dose expansion was conducted with 200 mg of Mab-1 Q3W (the fixed dose in selected tumors did not exceed the exposure of maximum tolerated dose) and the study was conducted in parallel with Phase 1B. All patients in phase 1B received Mab-1 as a 5 mg/kg IV infusion Q3W. Radiographic assessment was performed every 8 or 9 weeks per Response Evaluation Criteria In Solid Tumors guidelines version 1.1 (RECIST v1.1) and an example of this is shown in FIGS. 6A-6G.

### Key Eligibility Criteria of the UC Subset

Adult patients (aged ≥18 years) with histologically or cytologically confirmed UC who had at least one measurable lesion, as defined per RECIST v1.1; who had received standard therapy but no prior anti-PD-1 or programmed death-ligand 1 (PD-L1) treatment; and an Eastern Cooperative Oncology Group (ECOG) performance status of ≤1 were enrolled.

Patients were excluded if they had a history of severe hypersensitivity reactions to other antibodies. Patients who had prior malignancy active within the previous 2 years except for UC, and locally curable cancers that have been apparently cured, such as basal or squamous cell skin cancer, superficial bladder cancer, or carcinoma in situ of the cervix or breast, were also excluded.

Pretreatment tumor samples were retrospectively evaluated for PD-L1 membrane expression by immunohistochemistry performed on an automated platform. PD-L1 expression status was determined by PD-L1 membranous staining at any intensity on tumor cells (TC) or tumor-associated immune cells (IC). PD-L1 was defined as high if either ≥25% TC or ≥25% IC expressed membranous PD-L1, and PD-L1 was defined as low/negative if both TC and IC had <25% PD-L1 membrane staining. Eighteen (18) patients with UC (n=18) were enrolled in the clinical trials.

### Patient Disposition

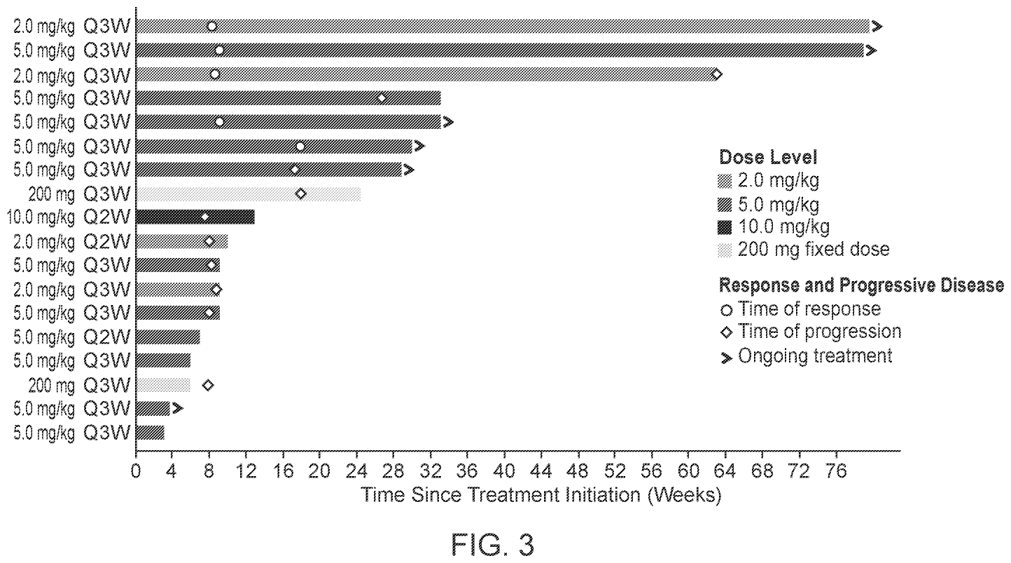

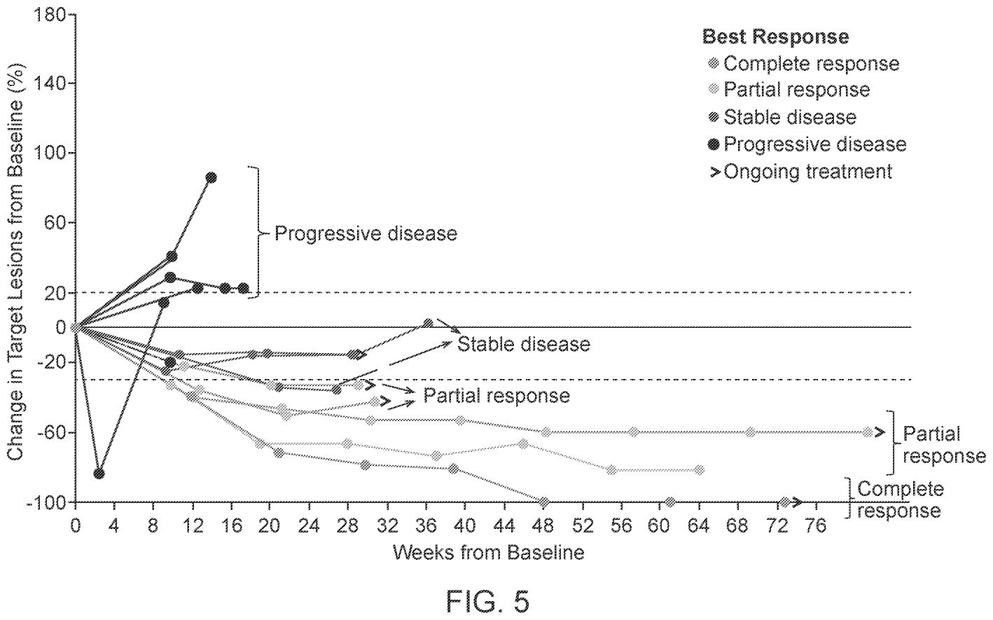

In the 18 enrolled patients with UC, 14 patients had at least one prior chemotherapy and six patients had prior radiotherapy indicating the difficulties in treating UC. A total of six patients (33.3%) remained on extended treatment with at least 3 patients receiving treatment past 36 weeks as shown in FIGS. 3 and 5. One of these patients showed complete response with the other two showing good partial response (FIG. 5). Table 1 shows the patient demographics and disease characteristics of patients with UC.

TABLE 1

| | UC Population (N = 18) | |
|---|---|---|
| Median age, years (min, max) | 71.5 | (39, 79) |

| | | |
|---|---|---|
| Sex, n | Male | 15 |
| | Female | 3 |
| Race, n | White | 18 |

| | | |
|---|---|---|
| Median treatment duration, months (min, max) | 2.6 | (0.7, 18.3) |

| | | |
|---|---|---|
| Prior anticancer | 1 | 7 |
| therapy regimens, n | | |
| | 2 | 3 |
| | ≥3 | 4 |

### Antitumor Activity

Among the enrolled 18 UC patients, 17 patients were evaluable. Evaluable UC patients are defined as having a measurable baseline tumor assessment and at least one evaluable post-baseline tumor response assessment, or had progressed or died prior to the initial tumor assessment.

In the clinical trials and results hereinafter, all patients received Mab-1 as a dose of 2.0, 5.0 or 10.0 mg/kg or a fixed dose of 200 mg IV infusion Q2W or Q3W.

Among the evaluable 17 patients with UC, one patient achieved a confirmed complete response (CR), four achieved a confirmed partial response (PR) and three achieved stable disease (SD). The disease control rate (DCR=CR+PR+SD) was found to be 47.1% (n=8/17). The median treatment duration was 3.0 months with the longest duration of 18.3 months and the shortest duration of 0.7 month. The antitumor activity of Mab-1 is presented in FIGS. 3 to 5.

### Response by PD-L1 Status

PD-L1 status and clinical response of the patients with UC treated with Mab-1 as a dose of 2.0, 5.0 or 10.0 mg/kg or a fixed dose of 200 mg IV infusion Q2W or Q3W were also evaluated.

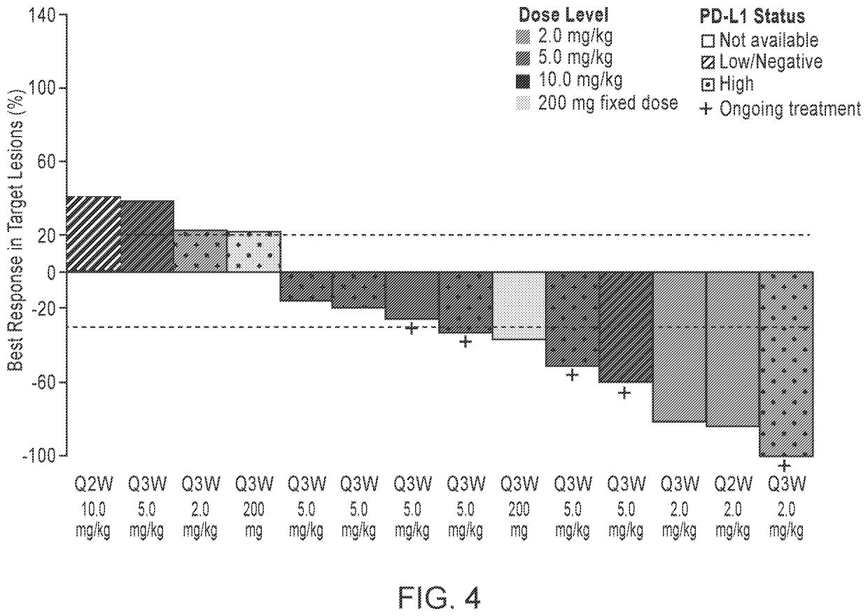

A total of 10 patients had both evaluable PD-L1 status and clinical responses. Clinical responses were observed in patients with both PD-L1 high and PD-L1 low expression (Table 2 and FIG. 4). Table 2 shows the confirmed best overall response for each evaluable patient by PD-L1 status.

TABLE 2
Confirmed Best Overall Response for Each Evaluable Patient by PD-L1 Status

| Patient | Treatment | Treatment | PD-L1 | Confirmed | Treatment |
|---|---|---|---|---|---|
| Number | Start | End | Status | Response | Ongoing |
| 1 | 19 Feb. 2016 | — | High | CR | Yes |
| 2 | 24 Jan. 2017 | 15 May 2017 | High | PD | No |
| 3 | 9 Mar. 2016 | 20 Apr. 2016 | High | PD | No |
| 4 | 29 Nov. 2016 | 31 Jan. 2017 | High | PD | No |
| 5 | 9 Jan. 2017 | — | High | PR | Yes |
| 6 | 31 Jan. 2017 | — | High | PR | Yes |
| 7 | 30 Jun. 2016 | 8 Mar. 2017 | High | SD | No |
| 8 | 23 Feb. 2016 | — | Low/negative | PR | Yes |
| 9 | 23 Feb. 2016 | 20 Apr. 2016 | Low/negative | PD | No |
| 10 | 11 Dec. 2015 | 3 Mar. 2016 | Low/negative | PD | No |
| 11 | 25 Nov. 2015 | 8 Feb. 2017 | Not available* | PR | No |
| 12 | 15 Sep. 2015 | 11 Nov. 2015 | Not available* | PD | No |
| 13 | 8 Feb. 2017 | — | Not available | SD | Yes |
| 14 | 14 Feb. 2017 | 2 Aug. 2017 | Not available | SD | No |

*PD-L1 status was not evaluable due to insufficient tumor tissue. PD-L1 high is defined as ≥25% tumor cells (TC) or ≥25% tumor-associated immune cells (IC); PD-L1 low/negative if both TC and IC with <25% PD-L1 staining.

The study found that the objective responses were observed at a higher rate in PD-L1⁺ disease compared with PD-L1⁻ disease, indicating that Mab-1 can be efficacious for patients with PD-L1⁺ UC.

### Safety and Tolerability

The clinical study also found that treatment with Mab-1 was generally well tolerated in patients with UC. Treatment-related AEs (TRAEs) occurred in 15 of the 18 patients with UC (Table 3), wherein fatigue (n=1), hyperglycemia (n=1), and type 1 diabetes mellitus (n=1), were the only AEs considered related to treatment that were Grade in severity regardless of attribution.

One patient discontinued treatment due to an infusion-related reaction considered related to Mab-1, and another patient had a treatment-emergent adverse event with a fatal outcome (muscle weakness), which was not related to Mab-1 treatment. This data is shown in Table 3.

TABLE 3
Treatment-Related AEs Occurring in ≥2 Patients with UC

UC Population (N = 18)

| | All grades, n | Grade ≥3, n |
|---|---|---|
| Fatigue | 5 | 1 |
| Rash | 3 | 0 |
| Nausea | 2 | 0 |
| Pain in extremity | 2 | 0 |
| Infusion-related reaction | 2 | 0 |

These results confirmed that the antibody disclosed herein (Mab-1) is tolerable and its toxicity profile demonstrates that adverse events (AEs) are generally low severity, manageable, and reversible.

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

What is claimed is:

1. A method of treatment of a patient with urothelial carcinoma comprising, administering to the patient a therapeutically effective amount of an anti-PD-1 antibody or an antigen binding fragment thereof, which reduces FcγR binding and reduces antibody-dependent phagocytosis.

2. The method of claim 1, wherein the anti-PD-1 antibody comprises a heavy chain variable region (V_{H}) and a light chain variable region (V_{L}) that contain the Complementarity Determining Regions (CDRs) set forth as follows:
a) mu317 CDR-H1, CDR-H2 and CDR-H3 (SEQ ID NOs: 11, 12, 13, respectively); and
CDR-L1, CDR-L2 and CDR-L3 (SEQ ID NOs: 14, 15, 16, respectively);
b) mu326 CDR-H1, CDR-H2 and CDR-H3 (SEQ ID NOs: 17, 18, 19, respectively); and
CDR-L1, CDR-L2 and CDR-L3 (SEQ ID NOs: 20, 21, 22, respectively);
c) 317-4B6 CDR-H1, CDR-H2 and CDR-H3 (SEQ ID NOs: 31, 32, 33, respectively); and
CDR-L1, CDR-L2 and CDR-L3 (SEQ ID NOs: 34, 35, 36, respectively);
d) 326-4A3 CDR-H1, CDR-H2 and CDR-H3 (SEQ ID NOs: 37, 38, 39, respectively); and
CDR-L1, CDR-L2 and CDR-L3 (SEQ ID NOs: 40, 41, 42, respectively);
e) 317-1H CDR-H1, CDR-H2 and CDR-H3 (SEQ ID NOs: 11, 59, 13, respectively); and
CDR-L1, CDR-L2 and CDR-L3 (SEQ ID NOs: 14, 15, 16, respectively);
f) 317-4B2 CDR-H1 CDR-H2 and CDR-H3 (SEQ ID NOs: 11, 60, 13, respectively); and
CDR-L1, CDR-L2 and CDR-L3 (SEQ ID NOs: 61 , 15, 16, respectively);
g) 317-4B5 CDR-H1, CDR-H2 and CDR-H3 (SEQ ID NOs: 11 , 60, 13, respectively); and
CDR-L1, CDR-L2 and CDR-L3 (SEQ ID NOs: 61 , 15, 16, respectively);
h) 317-4B6 CDR-H1, CDR-H2 and CDR-H3 (SEQ ID NOs: 11, 32, 13, respectively); and
CDR-L1, CDR-L2 and CDR-L3 (SEQ ID NOs: 61, 15, 16, respectively);
i) 326-1 CDR-H1, CDR-H2 and CDR-H3 (SEQ ID NOs: 17, 62, 19, respectively); and
CDR-L1, CDR-L2 and CDR-L3 (SEQ ID NOs: 20, 21, 22, respectively);
j) 326-3B1 CDR-H1, CDR-H2 and CDR-H3 (SEQ ID NOs: 17, 62, 19, respectively); and
CDR-LL CDR-L2 and CDR-L3 (SEQ ID NOs: 20, 21, 22, respectively); or
k) 326-3G1 CDR-H1, CDR-H2 and CDR-H3 (SEQ ID NOs: 17, 62, 19, respectively); and
CDR-L1, CDR-L 2 and CDR-L3 (SEQ ID NOs: 20, 21, 22, respectively).

3. The method of claim 1, wherein the anti-PD-1 antibody is an antibody comprising a heavy chain variable region (V_{H}) and a light chain variable region (V_{L}) that contain the CDRs set forth as follows:
(a) CDR-H1 (SEQ ID NO 31), CDR-H2 (SEQ ID NO 32,) and CDR-H3 (SEQ ID NO 33),
CDR-L1 (SEQ ID NO 34), CDR-L2 (SEQ ID NO 35) and CDR-L3 (SEQ ID NO 36); or
(b) CDR-H1 (SEQ ID NO 37), CDR-H2 (SEQ ID NO 38) and CDR-H3 (SEQ ID NO 39),
CDR-L1 (SEQ ID NO 40), CDR-L2 (SEQ ID NO 41) and CDR-L3 (SEQ ID NO 42).

4. The method of claim 1, wherein the anti-PD-1 antibody is an antibody which comprises a heavy chain variable region (V_{H}) and a light chain variable region (V_{L}) comprising:
a) mu317 (SEQ ID NOs: 4 and 6, respectively);
b) mu326 (SEQ ID NOs: 8 and 10, respectively);
c) 317-4B6 (SEQ ID NOs: 24 and 26, respectively);
d) 326-4A3 (SEQ ID NOs: 28 and 30, respectively);
e) 317-4B2 (SEQ ID NOs: 43 and 44, respectively);
f) 317-4B5 (SEQ ID NOs: 45 and 46, respectively);
g) 317-1 (SEQ ID NOs: 48 and 50, respectively);
h) 326-3B1 (SEQ ID NOs: 51 and 52, respectively);
i) 326-3GI (SEQ ID NOs: 53 and 54, respectively);
j) 326-1 (SEQ ID NOs: 56 and 58, respectively);
k) 317-3A1 (SEQ ID NOs: 64 and 26, respectively);
l) 317-3C1 (SEQ ID NOs: 65 and 26, respectively);
m) 317-3E1 (SEQ ID NOs: 66 and 26, respectively);
n) 317-3F1 (SEQ ID NOs: 67 and 26, respectively);
o) 317-3G1 (SEQ ID NOs: 68 and 26, respectively);
p) 317-3H1 (SEQ ID NOs: 69 and 26, respectively);
q) 317-311 (SEQ ID NOs: 70 and 26, respectively);
r) 317-4B 1 (SEQ ID NOs: 71 and 26, respectively);
s) 317-4B3 (SEQ ID NOs: 72 and 26, respectively);
t) 317-4B4 (SEQ ID NOs: 73 and 26, respectively);
u) 317-4A2 (SEQ ID NOs: 74 and 26, respectively);
v) 326-3 A 1 (SEQ ID NOs: 75 and 30, respectively);
w) 326-3C1 (SEQ ID NOs: 76 and 30, respectively);
x) 326-3D1 (SEQ ID NOs: 77 and 30, respectively);
y) 326-3E1 (SEQ ID NOs: 78 and 30, respectively);
z) 326-3F1 (SEQ ID NOs: 79 and 30, respectively);
aa) 326-3B N55D (SEQ ID NOs: 80 and 30, respectively);
ab) 326-4A1 (SEQ ID NOs: 28 and 81, respectively); or
ac) 326-4A2 (SEQ ID NOs: 28 and 82, respectively).

5. The method of any one of claims 2-4, wherein the anti-PD-1 antibody comprises a IgG4 heavy chain constant domain set forth in any one of SEQ ID NOs: 83-88 or 91-106.

6. The method of any one of claims 2-3, wherein the anti-PD-1 antibody which contains a F(ab) or F(ab′)2 comprising the CDRs of claim 2 or 3 or the heavy chain variable region (V_{H}) and the light chain variable region (V_{L}) of claim 4.

7. The method according to claim 1, wherein the anti-PD-1 antibody comprises a IgG4 heavy chain constant domain set forth in SEQ ID NOs: 87 or 88, and wherein the heavy chain variable region (V_{H}) and the light chain variable region (V_{L}) comprise:
a) mu317 (SEQ ID NOs: 4 and 6, respectively);
b) mu326 (SEQ ID NOs: 8 and 10, respectively);
c) 317-4B6 (SEQ ID NOs: 24 and 26, respectively);
d) 326-4A3 (SEQ ID NOs: 28 and 30, respectively);
e) 317-4B2 (SEQ ID NOs: 43 and 44, respectively);
f) 317-4B5 (SEQ ID NOs: 45 and 46, respectively);
g) 317-1 (SEQ ID NOs: 48 and 50, respectively);
h) 326-3B1 (SEQ ID NOs: 51 and 52, respectively);
i) 326-3GI (SEQ ID NOs: 53 and 54, respectively);
j) 326-1 (SEQ ID NOs: 56 and 58, respectively);
k) 317-3A1 (SEQ ID NOs: 64 and 26, respectively);
l) 317-3C1 (SEQ ID NOs: 65 and 26, respectively);
m) 317-3E1 (SEQ ID NOs: 66 and 26, respectively);
n) 317-3F1 (SEQ ID NOs: 67 and 26, respectively);
o) 317-3G1 (SEQ ID NOs: 68 and 26, respectively);
p) 317-3H1 (SEQ ID NOs: 69 and 26, respectively);
q) 317-311 (SEQ ID NOs: 70 and 26, respectively);
r) 317-4B 1 (SEQ ID NOs: 71 and 26, respectively);
s) 317-4B3 (SEQ ID NOs: 72 and 26, respectively);
t) 317-4B4 (SEQ ID NOs: 73 and 26, respectively);
u) 317-4A2 (SEQ ID NOs: 74 and 26, respectively);
v) 326-3 A 1 (SEQ ID NOs: 75 and 30, respectively);
w) 326-3C1 (SEQ ID NOs: 76 and 30, respectively);
x) 326-3D1 (SEQ ID NOs: 77 and 30, respectively);
y) 326-3E1 (SEQ ID NOs: 78 and 30, respectively);
z) 326-3F1 (SEQ ID NOs: 79 and 30, respectively);
aa) 326-3B N55D (SEQ ID NOs: 80 and 30, respectively);
ab) 326-4A1 (SEQ ID NOs: 28 and 81, respectively); or
ac) 326-4A2 (SEQ ID NOs: 28 and 82, respectively).

8. The method according to claim 1, wherein the anti-PD-1 antibody comprises an IgG4 heavy chain domain comprising SEQ ID NO: 88, and wherein the heavy chain variable region (V_{H}) and the light chain variable region (V_{L}) are set forth in SEQ ID NO: 24 and SEQ ID NO: 26, respectively.

9. The method of claim 1, wherein the anti-PD-1 antibody comprises an IgG4 Fc region comprising a S228P mutation at position 228 and amino acid mutations at positions 233, 234, and 235, wherein the mutations at positions 233, 234, and 235 reduce the binding to at least one Fcγ receptor relative to Fcγ binding of a reference IgG4 antibody having a mutation at position 228 only.

10. The method of claim 9, wherein the IgG4 Fc region comprises amino acid mutations at positions 228, 233, 234, 235, and 265.

11. The method of claim 9, wherein the IgG4 Fc region comprises amino acid mutations at positions 228, 233, 234, 235, 265, 309, and 409.

12. The method of claim 9, wherein the IgG4 Fc region consists of amino acid mutations of S228P, E233P, F234V, and L235A.

13. The method of claim 1, wherein the urothelial carcinoma is carcinoma of the ureter, urethra, renal pelvis and/or bladder.

14. The method of claim 13, wherein the urothelial carcinoma is transitional cell carcinoma.

15. The method of claim 13, wherein the urothelial carcinoma is advanced or metastatic.

16. The method of claim 13, wherein the urothelial carcinoma is PD-L1⁺ urothelial carcinoma or PD-L1⁻ urothelial carcinoma.

17. The method of claim 13, wherein the urothelial carcinoma is PD-L1⁺ urothelial carcinoma.

18. The method of claim 1, wherein the anti-PD-1 antibody is administered parenterally at a dose of 0.5-10 mg/kg QW, or Q2W, or Q3W, or Q4W.

19. The method of claim 18, wherein the anti-PD-1 antibody is administrated at a dose of 2-10 mg/kg QW or Q2W or Q3W.

20. The method of claim 18, wherein the anti-PD-1 antibody is administrated at a dose of 2-10 mg/kg or at a fixed dose of 200 mg Q2W or Q3W.

21. The method of claim 18, wherein the anti-PD-1 antibody is administrated intravenously at a dose of 2.0 mg/kg Q2W, 5 mg/kg Q2W, 2 mg/kg Q3W, or 5 mg/kg Q3W or at a fixed dose of 200 mg Q2W or Q3W.

22. The method of claim 1, further comprising administration of an additional therapeutic agent.

23. The method of claim 22, wherein the additional therapeutic agent is methotrexate, vinblastine, doxorubicin, and cisplatin (MVAC).

24. The method of claim 22, wherein the additional therapeutic agent is cisplatin.

25. The method of claim 24, wherein cisplatin is administered with gemcitabine.

26. The method of claim 22, wherein the additional therapeutic agent is paclitaxel.

27. The method of claim 25, wherein the paclitaxel is administered prior to administration of cisplatin and gemcitabine.


SEQUENCE LISTING (107 sequences)

SEQ ID NO: 1 - DNA, 444 bp, Homo sapiens
ccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaacc

SEQ ID NO: 2 - protein, 148 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQT

SEQ ID NO: 3 - DNA, 354 bp, Mus musculus
caggtgcagctgaaggagtcaggacctggcctggtggcgccctcaaagaacctgtccatcacttgcactgtctctgggttttcattaaccagctatggtgtacactggattcgccagcctccaggaaagggactggaatggctgggagtaatatgggccggtggaagcacaaattataattcggctctcatgtccagactgagcatcagcaaagacaactccaggagccaagttttcttaagaatgaacagtctgcaaactgatgacacagccatgtactactgtgccagagcctatggtaactactggtacatcgatgtctggggcgcagggaccacggtcaccgtctcctca

SEQ ID NO: 4 - protein, 118 aa, Mus musculus
QVQLKESGPGLVAPSKNLSITCTVSGFSLTSYGVHWIRQPPGKGLEWLGVIWAGGSTNYNSALMSRLSISKDNSRSQVFLRMNSLQTDDTAMYYCARAYGNYWYIDVWGAGTTVTVSS

SEQ ID NO: 5 - DNA, 321 bp, Mus musculus
gacattgtgatgacccagactcccaaattcctgcttgtatcagcaggagacagggttaccataacctgcaaggccagtcagagtgtgagtaatgatgtagcttggtaccaacagaagccagggcagtctcctaaactgctgataaactatgcatttcatcgcttcactggagtccctgatcgtttcactggcagtggatatgggacggatttcattttcaccatcagcactgtgcaggctgaagacctggcagtttatttctgtcaccaggcttatagttctccgtacacgttcggaggggggaccaagctggaaatgaaa

SEQ ID NO: 6 - protein, 107 aa, Mus musculus
DIVMTQTPKFLLVSAGDRVTITCKASQSVSNDVAWYQQKPGQSPKLLINYAFHRFTGVPDRFTGSGYGTDFIFTISTVQAEDLAVYFCHQAYSSPYTFGGGTKLEMK

SEQ ID NO: 7 - DNA, 342 bp, Mus musculus
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaataataatggagagccaacatatgctgaagagttcaagggacggtttgccttctctttggaaacctctgccagcactgcctatttgcagatcaacaacctcaaaaatgaggacacggctacatatttctgtgcaagagatgttatggactattggggtcaaggaacctcagtcaccgtctcctca

SEQ ID NO: 8 - protein, 114 aa, Mus musculus
QIQLVQSGPELKKPGETVKISCKASGYTFTNYGMNWVKQAPGKGLKWMGWINNNNGEPTYAEEFKGRFAFSLETSASTAYLQINNLKNEDTATYFCARDVMDYWGQGTSVTVSS

SEQ ID NO: 9 - DNA, 330 bp, Mus musculus
gacattgtgctgacccaatctccagcttctttggctgtgtctctagggcagagggccaccatatcctgcagagccagtgaaagtgttgataattatggctatagttttatgcactggtaccagcagaaaccaggacagccaccccaactcctcatctatcgtgcatccaacctagaatctgggatccctgccaggttcagtggcagtgggtctaggacaggcttcaccctcaccattaatcctgtggaggctgatgatgttgcaacctattactgtcagcaaagtaaagaatatccgacgttcggtggaggcaccaagctggaagtcaaa

SEQ ID NO: 10 - protein, 110 aa, Mus musculus
DIVLTQSPASLAVSLGQRATISCRASESVDNYGYSFMHWYQQKPGQPPQLLIYRASNLESGIPARFSGSGSRTGFTLTINPVEADDVATYYCQQSKEYPTFGGGTKLEVK

SEQ ID NO: 11 - protein, 10 aa, Mus musculus
GFSLTSYGVH

SEQ ID NO: 12 - protein, 16 aa, Mus musculus
VIWAGGSTNYNSALMS

SEQ ID NO: 13 - protein, 12 aa, Mus musculus
ARAYGNYWYIDV

SEQ ID NO: 14 - protein, 11 aa, Mus musculus
KASQSVSNDVA

SEQ ID NO: 15 - protein, 7 aa, Mus musculus
YAFHRFT

SEQ ID NO: 16 - protein, 9 aa, Mus musculus
HQAYSSPYT

SEQ ID NO: 17 - protein, 10 aa, Mus musculus
GYTFTNYGMN

SEQ ID NO: 18 - protein, 17 aa, Mus musculus
WINNNNGEPTYAEEFKG

SEQ ID NO: 19 - protein, 7 aa, Mus musculus
ARDVMDY

SEQ ID NO: 20 - protein, 15 aa, Mus musculus
RASESVDNYGYSFMH

SEQ ID NO: 21 - protein, 7 aa, Mus musculus
RASNLES

SEQ ID NO: 22 - protein, 8 aa, Mus musculus
QQSKEYPT

SEQ ID NO: 23 - DNA, 354 bp, Artificial Sequence
  317-4B6 cDNA-Vh
caggtgcagctgcaggagtcgggaccaggactggtgaagccttcggagaccctgtccctcacctgcactgtctctgggttttcattaaccagctatggtgtacactggatccggcagcccccagggaagggactggagtggatcggggtcatatacgccgatggaagcacaaattataatccctccctcaagagtcgagtgaccatatcaaaagacacctccaagaaccaggtttccctgaagctgagctctgtgaccgctgcggacacggccgtgtattactgtgcgagagcctatggtaactactggtacatcgatgtctggggccaagggaccacggtcaccgtctcctca

SEQ ID NO: 24 - protein, 118 aa, Artificial Sequence
  317-4B6 pro-Vh
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 25 - DNA, 321 bp, Artificial Sequence
  317-4B6 cDNA-Vk
gacatcgtgatgacccagtctccagactccctggctgtgtctctgggcgagagggccaccatcaactgcaagtccagcgagagtgtgagtaatgatgtagcttggtaccagcagaaaccaggacagcctcctaagctgctcattaactatgcatttcatcgcttcactggggtccctgaccgattcagtggcagcgggtatgggacagatttcactctcaccatcagcagcctgcaggctgaagatgtggcagtttattactgtcaccaggcttatagttctccgtacacgtttggccaggggaccaagctggagatcaaa

SEQ ID NO: 26 - protein, 107 aa, Artificial Sequence
  317-4B6 pro-Vk
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 27 - DNA, 342 bp, Artificial Sequence
  326-4A3 cDNA-Vh
caggtgcagctggtgcagagcggcagcgagctgaagaagcccggcgccagcgtgaaggtgagctgcaaggccagcggctacaccttcaccaactacggcatgaactgggtgagacaggcccccggccagggcctgaagtggatgggctggatcaacaacaacaacgccgagcccacctacgcccaggacttcagaggcagattcgtgttcagcctggacaccagcgccagcaccgcctacctgcagatcagcagcctgaagaccgaggacaccgccgtgtactactgcgccagagacgtgatggactactggggccagggcaccctggtgaccgtgagcagc

SEQ ID NO: 28 - protein, 114 aa, Artificial Sequence
  326-4A3 pro-Vh
QVQLVQSGSELKKPGASVKVSCKASGYTFTNYGMNWVRQAPGQGLKWMGWINNNNAEPTYAQDFRGRFVFSLDTSASTAYLQISSLKTEDTAVYYCARDVMDYWGQGTLVTVSS

SEQ ID NO: 29 - DNA, 330 bp, Artificial Sequence
  326-4A3 cDNA-Vk
gacattgtgctgacccagtctccagcctccttggccgtgtctccaggacagagggccaccatcacctgcagagccagtgaaagtgttgataattatggctatagttttatgcactggtatcagcagaaaccaggacaacctcctaaactcctgatttaccgtgcatccaacctagaatctggggtcccagccaggttcagcggcagtgggtctgggaccgatttcaccctcacaattaatcctgtggaagctgaggatactgcaaattattactgtcagcaaagtaaagaatatccgacgttcggcggagggaccaaggtggagatcaaa

SEQ ID NO: 30 - protein, 110 aa, Artificial Sequence
  326-4A3 pro-Vk
DIVLTQSPASLAVSPGQRATITCRASESVDNYGYSFMHWYQQKPGQPPKLLIYRASNLESGVPARFSGSGSGTDFTLTINPVEAEDTANYYCQQSKEYPTFGGGTKVEIK

SEQ ID NO: 31 - protein, 10 aa, Mus musculus
GFSLTSYGVH

SEQ ID NO: 32 - protein, 16 aa, Artificial Sequence
  317-4B6 H-CDR2 or CDR-H2
VIYADGSTNYNPSLKS

SEQ ID NO: 33 - protein, 12 aa, Mus musculus
ARAYGNYWYIDV

SEQ ID NO: 34 - protein, 11 aa, Artificial Sequence
  317-4B6 L-CDR1 or CDR-L1
KSSESVSNDVA

SEQ ID NO: 35 - protein, 7 aa, Mus musculus
YAFHRFT

SEQ ID NO: 36 - protein, 9 aa, Mus musculus
HQAYSSPYT

SEQ ID NO: 37 - protein, 10 aa, Mus musculus
GYTFTNYGMN

SEQ ID NO: 38 - protein, 17 aa, Artificial Sequence
  326-4A3 H-CDR2 or CDR-H2
WINNNNAEPTYAQDFRG

SEQ ID NO: 39 - protein, 7 aa, Mus musculus
ARDVMDY

SEQ ID NO: 40 - protein, 15 aa, Mus musculus
RASESVDNYGYSFMH

SEQ ID NO: 41 - protein, 7 aa, Mus musculus
RASNLES

SEQ ID NO: 42 - protein, 8 aa, Mus musculus
QQSKEYPT

SEQ ID NO: 43 - protein, 118 aa, Artificial Sequence
  317-4B2 pro-Vh
QVQLQESGPGLVKPSETLSLTCTVSGFSLTSYGVHWIRQPPGKGLEWIGVIYAGGSTNYNPSLKSRVTISKDTSKNQFSLKLSSVTAADTAVYYCARAYGNYWYIDVWGQGTTVTVSS

SEQ ID NO: 44 - protein, 107 aa, Artificial Sequence; identical to SEQ ID NO: 26
  317-4B2 pro-Vk

SEQ ID NO: 45 - protein, 118 aa, Artificial Sequence
  317-4B5 pro-Vh
QVQLQESGPGLVKPSETLSLTCTVSGFSLTSYGVHWIRQPPGKGLEWIGVIYAGGSTNYNPSLKSRVTISKDTSKNQVSLKLSSVTAADTAVYYCARAYGNYWYIDVWGQGTTVTVSS

SEQ ID NO: 46 - protein, 107 aa, Artificial Sequence; identical to SEQ ID NO: 26
  317-4B5 pro-Vk

SEQ ID NO: 47 - DNA, 354 bp, Artificial Sequence
  317-1 cDNA-Vh
caggtgcagctgcaggagtcgggaccaggactggtgaagccttcggagaccctgtccctcacctgcactgtctctgggttttcattaaccagctatggtgtacactggatccggcagcccccagggaagggactggagtggctgggggtcatatgggccggtggaagcacaaattataatccctccctcaagagtcgactgaccatatcaaaagacaactccaagagccaggtttccctgaagatgagctctgtgaccgctgcggacacggccgtgtattactgtgcgagagcctatggtaactactggtacatcgatgtctggggccaagggaccacggtcaccgtctcctca

SEQ ID NO: 48 - protein, 118 aa, Artificial Sequence
  317-1 pro-Vh
QVQLQESGPGLVKPSETLSLTCTVSGFSLTSYGVHWIRQPPGKGLEWLGVIWAGGSTNYNPSLKSRLTISKDNSKSQVSLKMSSVTAADTAVYYCARAYGNYWYIDVWGQGTTVTVSS

SEQ ID NO: 49 - DNA, 321 bp, Artificial Sequence
  317-1 cDNA-Vk
gacatcgtgatgacccagtctccagactccctggctgtgtctctgggcgagagggccaccatcaactgcaaggccagccagagtgtgagtaatgatgtagcttggtaccagcagaaaccaggacagcctcctaagctgctcattaactatgcatttcatcgcttcactggggtccctgaccgattcagtggcagcgggtatgggacagatttcactctcaccatcagcagcctgcaggctgaagatgtggcagtttattactgtcaccaggcttatagttctccgtacacgtttggcggggggaccaagctggagatcaaa

SEQ ID NO: 50 - protein, 107 aa, Artificial Sequence
  317-1 pro-Vk
DIVMTQSPDSLAVSLGERATINCKASQSVSNDVAWYQQKPGQPPKLLINYAFHRFTGVPDRFSGSGYGTDFTLTISSLQAEDVAVYYCHQAYSSPYTFGGGTKLEIK

SEQ ID NO: 51 - protein, 114 aa, Artificial Sequence
  326-3B1 pro-Vh
QVQLVQSGSELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTLVTVSS

SEQ ID NO: 52 - protein, 110 aa, Artificial Sequence
  326-3B1 pro-Vk
DIVLTQSPASLAVSPGQRATITCRASESVDNYGYSFMHWYQQKPGQPPKLLIYRASNLESGVPARFSGSGSRTDFTLTINPVEANDTANYYCQQSKEYPTFGGGTKVEIK

SEQ ID NO: 53 - protein, 114 aa, Artificial Sequence
  326-3G1 pro-Vh
QVQLVQSGPELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTLVTVSS

SEQ ID NO: 54 - protein, 110 aa, Artificial Sequence; identical to SEQ ID NO: 52
  326-3G1 pro-Vk

SEQ ID NO: 55 - DNA, 342 bp, Artificial Sequence
  326-1 cDNA-Vh
caggtgcagctggtgcagagcggcagcgagctgaagaagcccggcgccagcgtgaaggtgagctgcaaggccagcggctacaccttcaccaactacggcatgaactgggtgagacaggcccccggccagggcctggagtggatgggctggatcaacaacaacaacggcgagcccacctacgcccagggcttcagaggcagattcgtgttcagcctggacaccagcgccagcaccgcctacctgcagatcagcagcctgaagaccgaggacaccgccgtgtacttctgcgccagagacgtgatggactactggggccagggcaccaccgtgaccgtgagcagc

SEQ ID NO: 56 - protein, 114 aa, Artificial Sequence
  326-1 pro-Vh
QVQLVQSGSELKKPGASVKVSCKASGYTFTNYGMNWVRQAPGQGLEWMGWINNNNGEPTYAQGFRGRFVFSLDTSASTAYLQISSLKTEDTAVYFCARDVMDYWGQGTTVTVSS

SEQ ID NO: 57 - DNA, 330 bp, Artificial Sequence
  326-1 cDNA-Vk
gacattgtgctgacccagtctccagcctccttggccgtgtctccaggacagagggccaccatcacctgcagagccagtgaaagtgttgataattatggctatagttttatgcactggtatcagcagaaaccaggacaacctcctaaactcctgatttaccgtgcatccaacctagaatctggggtcccagccaggttcagcggcagtgggtctaggaccgatttcaccctcacaattaatcctgtggaagctaatgatactgcaaattattactgtcagcaaagtaaagaatatccgacgttcggcggagggaccaaggtggagatcaaa

SEQ ID NO: 58 - protein, 110 aa, Artificial Sequence; identical to SEQ ID NO: 52
  326-1 pro-Vk

SEQ ID NO: 59 - protein, 16 aa, Artificial Sequence
  317-1 H-CDR2 or CDR-H2
VIWAGGSTNYNPSLKS

SEQ ID NO: 60 - protein, 16 aa, Artificial Sequence
  317-4B2 H-CDR2 or CDR-H2
VIYAGGSTNYNPSLKS

SEQ ID NO: 61 - protein, 11 aa, Artificial Sequence
  317-4B2 L-CDR1 or CDR-L1
KSSESVSNDVA

SEQ ID NO: 62 - protein, 17 aa, Artificial Sequence
  326-1 H-CDR2 or CDR-H2
WINNNNGEPTYAQGFRG

SEQ ID NO: 63 - protein, 17 aa, Artificial Sequence
  326-3G1 H-CDR2 or CDR-H2
WINNNNGEPTYAQDFRG

SEQ ID NO: 64 - protein, 118 aa, Artificial Sequence
  317-3A1 pro-Vh
QVQLQESGPGLVKPSETLSLTCTVSGFSLTSYGVHWIRQPPGKGLEWIGVIWAGGSTNYNPSLKSRVTISVDTSKNQFSLKLSSVTAADTAVYYCARAYGNYWYIDVWGQGTTVTVSS

SEQ ID NO: 65 - protein, 118 aa, Artificial Sequence
  317-3C1 pro-Vh
QVQLQESGPGLVKPSETLSLTCTVSGFSLTSYGVHWIRQPPGKGLEWLGVIWAGGSTNYNPSLKSRVTISVDTSKNQFSLKLSSVTAADTAVYYCARAYGNYWYIDVWGQGTTVTVSS

SEQ ID NO: 66 - protein, 118 aa, Artificial Sequence
  317-3E1 pro-Vh
QVQLQESGPGLVKPSETLSLTCTVSGFSLTSYGVHWIRQPPGKGLEWIGVIWAGGSTNYNPSLKSRVTISVDTSKSQFSLKLSSVTAADTAVYYCARAYGNYWYIDVWGQGTTVTVSS

SEQ ID NO: 67 - protein, 118 aa, Artificial Sequence
  317-3F1 pro-Vh
QVQLQESGPGLVKPSETLSLTCTVSGFSLTSYGVHWIRQPPGKGLEWIGVIWAGGSTNYNPSLKSRVTISVDTSKNQVSLKLSSVTAADTAVYYCARAYGNYWYIDVWGQGTTVTVSS

SEQ ID NO: 68 - protein, 118 aa, Artificial Sequence
  317-3G1 pro-Vh
QVQLQESGPGLVKPSETLSLTCTVSGFSLTSYGVHWIRQPPGKGLEWIGVIWAGGSTNYNPSLKSRVTISVDTSKSQVSLKLSSVTAADTAVYYCARAYGNYWYIDVWGQGTTVTVSS

SEQ ID NO: 69 - protein, 118 aa, Artificial Sequence
  317-3H1 pro-Vh
QVQLQESGPGLVKPSETLSLTCTVSGFSLTSYGVHWIRQPPGKGLEWIGVIWAGGSTNYNPSLKSRVTISKDTSKSQVSLKLSSVTAADTAVYYCARAYGNYWYIDVWGQGTTVTVSS

SEQ ID NO: 70 - protein, 118 aa, Artificial Sequence
  317-3I1 pro-Vh
QVQLQESGPGLVKPSETLSLTCTVSGFSLTSYGVHWIRQPPGKGLEWIGVIWAGGSTNYNPSLKSRVTISKDTSKNQVSLKLSSVTAADTAVYYCARAYGNYWYIDVWGQGTTVTVSS

SEQ ID NO: 71 - protein, 118 aa, Artificial Sequence
  317-4B1 pro-Vh
QVQLQESGPGLVKPSETLSLTCTVSGFSLTSYGVHWIRQPPGKGLEWIGVIWAGGSTNYNPSLKSRVTISKDTSKNQFSLKLSSVTAADTAVYYCARAYGNYWYIDVWGQGTTVTVSS

SEQ ID NO: 72 - protein, 118 aa, Artificial Sequence
  317-4B3 pro-Vh
QVQLQESGPGLVKPSETLSLTCTVSGFSLTSYGVHWIRQPPGKGLEWIGVINAGGSTNYNPSLKSRVTISKDTSKNQFSLKLSSVTAADTAVYYCARAYGNYWYIDVWGQGTTVTVSS

SEQ ID NO: 73 - protein, 118 aa, Artificial Sequence
  317-4B4 pro-Vh
QVQLQESGPGLVKPSETLSLTCTVSGFSLTSYGVHWIRQPPGKGLEWIGVIWAGGSTNYNPSLKSRVTISKDTSKNQFSLKLSSVTAADTAVYYCARAYGNYPYIDVWGQGTTVTVSS

SEQ ID NO: 74 - protein, 107 aa, Artificial Sequence
  317-4A2 pro-Vk
DIVMTQSPDSLAVSLGERATINCKSSQSVSNDVAWYQQKPGQPPKLLINYAFHRFTGVPDRFSGSGYGTDFTLTISSLQAEDVAVYYCHQAYSSPYTFGQGTKLEIK

SEQ ID NO: 75 - protein, 114 aa, Artificial Sequence
  326-3A1 pro-Vh
QVQLVQSGPELKKPGASVKVSCKASGYTFTNYGMNWVRQAPGQGLEWMGWINNNNGEPTYAQGFRGRFVFSLDTSASTAYLQISSLKTEDTAVYYCARDVMDYWGQGTLVTVSS

SEQ ID NO: 76 - protein, 114 aa, Artificial Sequence
  326-3C1 pro-Vh
QVQLVQSGPELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTTVTVSS

SEQ ID NO: 77 - protein, 114 aa, Artificial Sequence
  326-3D1 pro-Vh
QVQLVQSGSELKKPGASVKVSCKASGYTFTNYGMNWVRQAPGQGLEWMGWINNNNGEPTYAQGFRGRFVFSLDTSASTAYLQISSLKTEDTAVYYCARDVMDYWGQGTLVTVSS

SEQ ID NO: 78 - protein, 114 aa, Artificial Sequence
  326-3E1 pro-Vh
QVQLVQSGSELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTTVTVSS

SEQ ID NO: 79 - protein, 114 aa, Artificial Sequence
  326-3F1 pro-Vh
QVQLVQSGPELKKPGASVKVSCKASGYTFTNYGMNWVRQAPGQGLEWMGWINNNNGEPTYAQGFRGRFVFSLDTSASTAYLQISSLKTEDTAVYYCARDVMDYWGQGTTVTVSS

SEQ ID NO: 80 - protein, 114 aa, Artificial Sequence
  326-3B N55D pro-Vh
QVQLVQSGSELKKPGASVKVSCKASGYTFTNYGMNWVRQAPGQGLKWMGWINNNDGEPTYAQDFRGRFVFSLDTSASTAYLQISSLKTEDTAVYYCARDVMDYWGQGTLVTVSS

SEQ ID NO: 81 - protein, 110 aa, Artificial Sequence
  326-4A1 pro-Vk
DIVLTQSPASLAVSPGQRATITCRASESVDNYGYSFMHWYQQKPGQPPKLLIYRASNLESGVPARFSGSGSRTDFTLTINPVEAEDTANYYCQQSKEYPTFGGGTKVEIK

SEQ ID NO: 82 - protein, 110 aa, Artificial Sequence
  326-4A2 pro-Vk
DIVLTQSPASLAVSPGQRATITCRASESVDNYGYSFMHWYQQKPGQPPKLLIYRASNLESGVPARFSGSGSGTDFTLTINPVEANDTANYYCQQSKEYPTFGGGTKVEIK

SEQ ID NO: 83 - protein, 327 aa, Artificial Sequence
  huIgG4mt1 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 327 aa, Artificial Sequence
  huIgG4mt2 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 327 aa, Artificial Sequence
  huIgG4mt6 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 327 aa, Artificial Sequence
  huIgG4mt8 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVTVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 327 aa, Artificial Sequence
  huIgG4mt9 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 88 - protein, 327 aa, Artificial Sequence
  huIgG4mt10 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 89 - protein, 367 aa, Artificial Sequence
  OS8 pro
MERHWIFLLLLSVTAGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIV

SEQ ID NO: 90 - protein, 304 aa, Artificial Sequence
  P3Z pro
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 91 - protein, 327 aa, Artificial Sequence
  IgG4 Fc region 228P-233P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPEFGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - protein, 327 aa, Artificial Sequence
  IgG4 Fc region 228P-234V
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEVFGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 327 aa, Artificial Sequence
  IgG4 Fc region 228P-235A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 327 aa, Artificial Sequence
  IgG4 Fc region 228P-234V-235A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 327 aa, Artificial Sequence
  IgG4 Fc region 228P-234A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAFGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 96 - protein, 327 aa, Artificial Sequence
  IgG4 Fc region 228P-234A-235A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 327 aa, Artificial Sequence
  IgG4 Fc region 228P-233P-234A-235A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 327 aa, Artificial Sequence
  IgG4 Fc region 228P-234V-235A-265A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEVAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 327 aa, Artificial Sequence
  IgG4 Fc region 228P-234A-235A-265A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 327 aa, Artificial Sequence
  IgG4 Fc region 228P-233P-234A-235A-265A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPAAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 327 aa, Artificial Sequence
  IgG4 Fc region 228P-265A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 327 aa, Artificial Sequence
  IgG4 Fc region 228P-309V
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - protein, 327 aa, Artificial Sequence
  IgG4 Fc region 228P-409K
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 104 - protein, 327 aa, Artificial Sequence
  IgG4 Fc region 228P-309V-409K
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 105 - protein, 327 aa, Artificial Sequence
  IgG4 Fc region 228P-265A-309V-409K
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 106 - protein, 327 aa, Artificial Sequence
  IgG4 Fc region 228P-233P-234A-235A-265A-309V- 409K
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPAAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 107 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK